(19) United States
(12) Patent Application Publication
(10) Pub. No.: US 2010/0209495 A1
(43) Pub. Date: Aug. 19, 2010

(54) GRANULATES, PROCESS FOR PREPARING THEM AND PHARMACEUTICAL PRODUCTS CONTAINING THEM

(75) Inventors: Boyong LI, Morgantown, WV (US); Thomas Daniel Reynolds, Morgantown, WV (US)

Correspondence Address: BUCHANAN, INGERSOLL & ROONEY PC, POST OFFICE BOX 1404, ALEXANDRIA, VA, 22313-1404, US

(73) Assignee: Mylan Laboratories, Inc., Morgantown, WV (US)

(21) Appl. No.: 12/560,668

(22) Filed: Sep. 16, 2009

Related U.S. Application Data
(60) Provisional application No. 61/097,667, filed on Sep. 17, 2008.

(51) Int. Cl. A61K 31/165; A61K 9/48; A61K 9/14; A61K 9/20; A61P 35/00

(52) U.S. Cl. 424/452; 514/618; 424/451; 424/494; 424/497; 424/490; 424/465; 424/464

(57) ABSTRACT

A granulate for use in a pharmaceutical composition and a pharmaceutical composition manufacture using the granulate, where the granule comprises an active pharmaceutical ingredient (API) having a poor water solubility (i.e., less than about 1 mg/mL) which is intimately associated with at least one pharmaceutically acceptable hydrophilic polymer. The granule optionally contains one or more pharmaceutically acceptable excipients, such as disintegrants, wetting agents, diluents, binders, lubricants, glidants, coloring agents and flavoring agents. The invention also relates to a process for preparing the pharmaceutical granulate and pharmaceutical compositions containing the granulate.

### CROSS-REFERENCE TO PRIORITY/PROVISIONAL APPLICATION

This application claims priority under 35 U.S.C. §120 of U.S. Provisional Application No. 61/097,667, filed Sep. 17, 2008, hereby expressly incorporated by reference and assigned to the assignee hereof.

### FIELD OF THE INVENTION

The present invention relates to granules having a core containing an active pharmaceutical ingredient that itself has poor aqueous solubility where the active pharmaceutical ingredient is intimately associated with one or more hydrophilic polymers. The granules are useful in the manufacture of pharmaceutical compositions, as exemplified by formulations of bicalutamide.

### BACKGROUND OF THE INVENTION

The aqueous solubility of an active pharmaceutical ingredient (“API”) influences both the bioavailability of the drug and the rate at which the API can be released from a formulated product. The rate of dissolution of an API from a formulation can place an upper limit on the rate of absorption of the API in a person to whom the product is administered. Many active pharmaceutical ingredients have poor aqueous solubility and low bioavailability. One method that has been used to improve the dissolution of API's is to reduce the particles size of the active ingredient, which increases the surface area of the active ingredient and may result in an increased rate of dissolution. This approach is limited by the particle size that can be achieved and by poor bulk flow and handling characteristics of finely powdered active pharmaceutical ingredients, which often require special isolation and handling procedures due to the toxicological nature of many API's. One method for improving the dissolution rate involves spray drying a solution of the API and hydrophilic polymers. (See, Marc Hugo, et al.: Dissolution rate of poorly soluble fenofebrate can be improved by solid dispersion in hydrophilic polymers using spray drying. AAPS Annual Meeting and Exposition, Oct. 2, 2006. San Antonio, Tex.). This approach is limited by the resulting fine powder material that has poor bulk flow and handling characteristics and the need for extra processing steps to make solid dosage forms such as tablets and capsules. Another process for enhancing dissolution involves kneading a mixture of an API with polyvinyl pyrrolidone dispersed in water, drying the paste, to form a powder. The powder is then screened and compressed into tablets. (See, Aftab Modi and Pralhad Tayade: Enhancement of dissolution profile by solid dispersion (kneading) technique. AAPS PharmSciTech 2006. 7(3), Article 68). The kneading process is limited by the special difficulties involved in drying the paste material.

The standard one-step granulation process commonly used in the pharmaceutical industry produces granules by adding a solution or mixture of a drug and an excipient, such as a binder, to a solid mixture of other excipients. When the drug substance has poor aqueous solubility, this process is found to be unsuitable because the granules formed include large agglomerates and the time duration of the granulation process has a sharp endpoint. In addition, although tablets made from a one-step wet granulation process show acceptable dissolution characteristics, these tablets are observed to erode unevenly during dissolution testing. These observed processing and dissolution characteristics are believed to be related to a non-uniform distribution of the binder, such as povidone, which acts as a wetting agent and the inadequate wetting of the drug substance during granulation. When the drug substance has poor aqueous solubility, the standard wet granulation process also results in inadequate and uneven contact between the drug and hydrophilic polymer.

Many API's have low aqueous solubility. An example of an API with low aqueous solubility is bicalutamide, which has a water solubility of 5 mg/1000 ml at 37° C. Bicalutamide is the common name for the compound N-[4-cyano-3-(trifluoromethyl)phenyl]-3-(4-fluorophenyl) sulfonyl-2-hydroxy-2-methyl-propanamide, which is also known as 4′-cyano-3-((4-fluorophenyl)sulfonyl)-2-hydroxy-2-methyl-3′-(trifluoromethyl)propionanilide. The structure of bicalutamide is shown in formula (I) below:

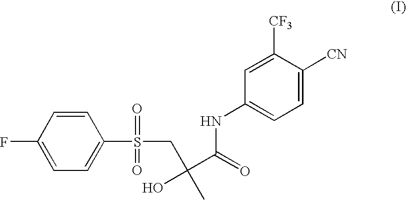

Bicalutamide is an oral non-steroidal anti-androgen used for the treatment of prostate cancer and hirsutism.

### SUMMARY OF THE INVENTION

To address these process and dissolution issues, the process described herein, termed “reverse wet granulation” was developed. In the process, the API is intimately mixed with a solution or suspension of a hydrophilic polymer to form a drug-polymer slurry. Granules can then be formed by incorporating a mixture of other dry excipients into the drug-polymer slurry. The granules formed comprises a core containing an active pharmaceutical ingredient that itself has poor aqueous solubility where the active pharmaceutical ingredient is intimately associated with one or more hydrophilic polymers. The term granulate, when used as a noun, refers to group of granules. Granules produced by this process, after milling, have good flow and handling characteristics like those produced with the commonly used one-step process. However, tablets formed from these granules erode more uniformly during dissolution testing.

According to one aspect of the invention, granules for use in a pharmaceutical composition comprises a core comprising at least one active pharmaceutical ingredient intimately associated with at least one hydrophilic polymer, where the active pharmaceutical ingredient has a solubility in water of less than about 1 mg/ml.

According to another aspect of the invention, the granule further comprises at least one excipient selected from the group consisting of diluents, disintegrants, binders, wetting agents, lubricants, glidants, coloring agents and flavoring agents.

According to yet another aspect of the invention, a pharmaceutical dosage form comprises a granule which comprises a core with at least one active pharmaceutical ingredient intimately associated with at least one hydrophilic polymer, where the active pharmaceutical ingredient has a solubility in water of less than about 1 mg/ml.

According to still another aspect of the invention, a process for making a granulate comprises the steps of:

(a) dissolving or suspending at least one hydrophilic polymer in a solvent to form a solution or suspension, respectively,

(b) combining the solution formed in step (a) with an active pharmaceutical ingredient having a solubility in water of less than about 1 mg/ml to form a mixture and blending the mixture to form a slurry,

(c) combining the mixture formed in step (b) with an excipient or a mixture of excipients to form a wet granulate, and

(d) drying the wet granulate to obtain a dry granulate where the wet granulate formed in step (c) comprises a core containing an active pharmaceutical ingredient that itself has poor aqueous solubility where the active pharmaceutical ingredient is intimately associated with one or more hydrophilic polymers.

According to another aspect of the invention, the process for making a granulate further comprises the step of milling the dry granulate to obtain a modified dry granulate having a desired particle size distribution.

According to yet another aspect of the invention, a process for making a pharmaceutical tablet comprises compressing a granulate comprising at least one active pharmaceutical ingredient intimately associated with at least one hydrophilic polymer, where the active pharmaceutical ingredient has a solubility in water of less than about 1 mg/ml, into tablets.

According to still another aspect of the invention, the granulate is a composition produced by a process comprising the steps of:

(a) forming a mixture core by (1) dissolving or suspending at least one hydrophilic polymer in a solvent to form a solution or suspension, respectively, and (2) combining the solution or suspension formed in step (1) with an active pharmaceutical ingredient having a solubility in water of less than about 1 mg/ml to form a mixture and blending the mixture to form a slurry,

(b) combining the mixture formed in step (a) with an excipient or a mixture of excipients to form a wet granulate, and

(c) drying the wet granulate to obtain a dry granulate.

### BRIEF DESCRIPTION OF THE DRAWING

FIG. 1 is a representation of the granule of the invention.

FIG. 2 depicts a process for manufacturing the granulate by reverse wet granulation.

### DETAILED DESCRIPTION OF THE INVENTION

The present invention provides granules comprising having an active pharmaceutical ingredient having poor aqueous solubility and methods for making such granules. The granules are useful for making oral solid dosage forms, for example capsules and compressed tablets, in a variety of shapes and sizes. The advantages of the present inventive composition and method are notable with active pharmaceutical ingredients that have poor aqueous solubility.

In this application, the term “active pharmaceutical ingredient, which has poor aqueous solubility” or “active pharmaceutical ingredient having poor aqueous solubility” means an API or drug having a solubility in water of less than about 1 mg/mL, i.e., the compound is water insoluble (<0.1 mg/mL) or very slightly soluble (0.1-1.0 mg/mL), according to the USP definition of solubility. Examples of an “active pharmaceutical ingredient, which has poor aqueous solubility” or an “active pharmaceutical ingredient having poor aqueous solubility” include anastrozole, aripiprazole, atorvastatin, bicalutamide, candesartan, celecoxib, dutasteride, ezetimibe, fenofibrate, glyburide, meloxicam, oxcarbazepine, raloxifene, rifaximine, rofecoxib, simvastatin, and valdecoxib. In a preferred embodiment, the active pharmaceutical ingredient is bicalutamide. The granules described herein can be used with racemic mixtures of an active ingredient having poor aqueous solubility or with individual isomers of such active ingredient. The granules can comprise one or more active ingredients where at least one of the active ingredients has poor aqueous solubility.

In the granules of the invention, the active pharmaceutical ingredient having poor aqueous solubility and the one or more pharmaceutically acceptable hydrophilic polymers are intimately associated or are in intimate association. The term “intimately associated” or “intimate association” refers to a state produced by a process comprising mixing the API and a solution of the one or more pharmaceutically acceptable hydrophilic polymers to form a mixture in the form of a slurry. The API and the one or more pharmaceutically acceptable hydrophilic polymers are intimately associated or in intimate association, with the hydrophilic polymer providing a coating on particles of the API. Granules are formed by mixing one or more excipients with the intimate association of the API and the at least one hydrophilic polymer. This subunit of the granule is termed the drug dispersion.

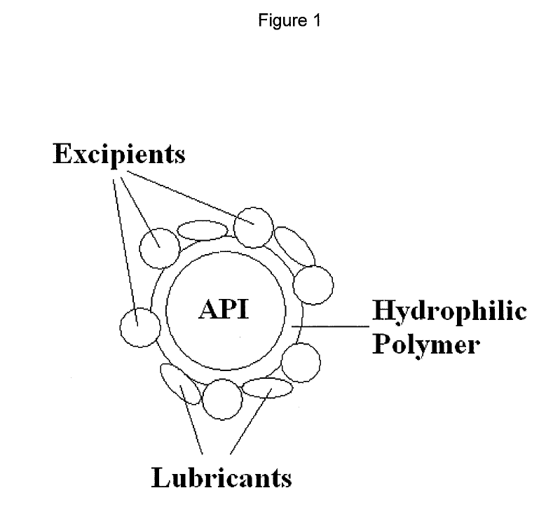

Granules of this invention are represented in FIG. 1. The granule comprises a core to which one or more excipients are in contact or attached. The core comprises at least one API and the one or more pharmaceutically acceptable hydrophilic polymers, where the one or more pharmaceutically acceptable hydrophilic polymers coat particles of the API. One or more excipients are in contact or attached to the core. The granule may also include additional excipients, as exemplified by one or more lubricants, which are added to the granule in a further processing step.

The core of the granule, where the API and the one or more pharmaceutically acceptable hydrophilic polymers are in intimate association, achieves a consistency and stable adherence between the API and the at least one hydrophilic polymer. As a result of the intimate association between the API and the at least one hydrophilic polymer, pharmaceutical compositions produced using granules of the invention have more uniform wetting and dissolution.

Examples of pharmaceutically acceptable hydrophilic polymers include polyvinyl pyrrolidone (also known as PVP or povidone), hydroxypropyl methylcellulose, hydroxypropyl cellulose, polyethylene glycol, hydroxyethyl cellulose, polyethylene oxide, carbomer, polyvinyl alcohol, and/or mixtures thereof. In one particular embodiment, the hydrophilic polymer is povidone.

The granule may further comprise at least one excipient in addition to the one or more pharmaceutically acceptable hydrophilic polymers. Such excipients include diluents, disintegrants, binders, wetting agents, lubricants, glidants, coloring agents and flavoring agents. Examples of such excipients are well known to one skilled in the art. See for example Rowe, et al., Handbook of Pharmaceutical Excipients, 5th Edition, which is hereby incorporated by reference in its entirety. In one embodiment, the at least one excipient comprises at least one diluent and at least one disintegrant. Examples of diluents include lactose monohydrate, microcrystalline cellulose, calcium phosphate dibasic, sucrose, mannitol, starch, pregelatinized starch, lactose, sorbitol, glucose, fructose, galactose, maltose, isomaltose, aluminum oxide, bentonite, powdered cellulose, kaolin, magnesium carbonate, saponite, and mixtures thereof. In one particular embodiment, the diluent is lactose monohydrate. Examples of disintegrants include crospovidone, croscarmellose sodium, sodium starch glycolate, and mixtures thereof. In an embodiment the disintegrant is sodium starch glycolate. Examples of lubricants include magnesium stearate, sodium lauryl sulfate, colloidal silicon dioxide, calcium stearate, magnesium lauryl sulfate, potassium benzoate, sodium benzoate, talc, zinc stearate, sodium stearyl fumarate and mixtures thereof. In one embodiment, the lubricant is a mixture of magnesium stearate and sodium lauryl sulfate. In another embodiment, the lubricant is colloidal silicon dioxide. In yet another embodiment, both colloidal silicon dioxide and a mixture of magnesium stearate and sodium lauryl sulfate are used together.

In one embodiment, the granule comprises bicalutamide as the active pharmaceutical ingredient and povidone as the at least one hydrophilic polymer. In another embodiment, the granule comprises bicalutamide as the active pharmaceutical ingredient, povidone as the at least one hydrophilic polymer, and lactose monohydrate and sodium starch glycolate as excipients. In still another embodiment, the granule comprises bicalutamide as the active pharmaceutical ingredient, povidone as the at least one hydrophilic polymer, and lactose monohydrate, sodium starch glycolate and povidone as excipients. In a further embodiment, the granule comprises bicalutamide as the active pharmaceutical ingredient, povidone as the at least one hydrophilic polymer, and lactose monohydrate, sodium starch glycolate and povidone as excipients, and colloidal silicon dioxide and a mixture of magnesium stearate and sodium lauryl sulfate as lubricants. In another embodiment, the granule further comprises at least one hydrophilic polymer that is not intimately associated with the active ingredient. The weight ratio of the API to the at least one hydrophilic polymer in the granule is preferably at least 1:10, more preferably at least 1:5, and even more preferably at least 1:1, and is preferably less than 50:1, more preferably less than 20:1 and even more preferably less than 10:1.

The granule described in the various embodiments above can be used in pharmaceutical dosage forms, such as tablets and capsules. In one embodiment, the dosage form is a tablet and the API comprises bicalutamide. In another embodiment, the dosage form is a tablet, the API comprises bicalutamide and the at least one hydrophilic polymer is povidone. In yet another embodiment, the dosage form comprises granules which comprise at least one diluent, at least one disintegrant and at least one lubricant. In still another embodiment, the dosage form comprise granules which comprise lactose monohydrate, as the diluent, sodium starch glycolate as the disintegrant and a mixture of magnesium stearate/sodium lauryl sulfate as the lubricant. In a further embodiment, the dosage form comprise granules which comprise lactose monohydrate, as the diluent, sodium starch glycolate as the disintegrant and both colloidal silicon dioxide and a mixture of magnesium stearate/sodium lauryl sulfate as the lubricant.

The solid pharmaceutical formulations, e.g., tablets and capsules, of the present invention can display dissolution properties that can be adjusted to obtain a desired profile by altering the specific excipients used within the capsules or tablets as well as by altering the nature and/or quantity of a coating on the tablets. Altering the nature and/or quantity of the excipients in a tablet or capsule and/or a coating on a tablet to obtain a desired release rate can be performed using methods known to one of ordinary skill in the art. In one embodiment, minitablets comprising the granule described herein may be contained within capsules. A capsule may contain minitablets having essentially a uniform release rate or may contain minitablets having different release rates. Methods of adjusting the overall release rate of an active ingredient from a capsule using a plurality of minitablets having different individual release rates are known to one of ordinary skill in the art.

In one embodiment, the pharmaceutical dosage form is a tablet comprising granules which comprise bicalutamide as the API, wherein the amount of bicalutamide dissolves in the time listed below when tested under conditions described as the USP apparatus II (paddle) test, using 1000 ml of a 1% aqueous solution of sodium lauryl sulfate at 37° C. with the paddle apparatus rotating at 50 rpm.

| approximate | approximate |
|---|---|
| % dissolved | time (minutes) |
| 30 | 5 |
| 70 | 15 |
| 95 | 30 |
| 97 | 45 |
| 99 | 60 |

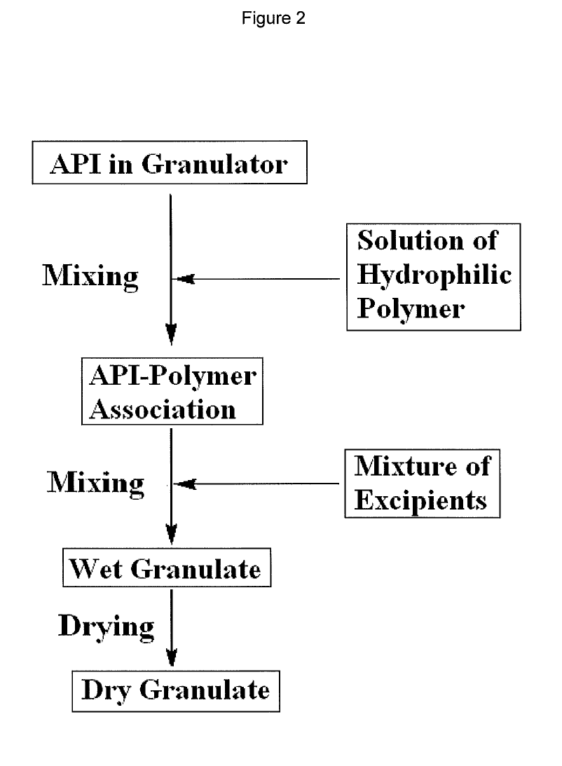

The present invention also relates to a process for making a granulate for use in a pharmaceutical composition which is an oral solid dosage form. The process is outlined in FIG. 2. The process comprises the steps of: (a) forming a slurry of drug dispersion ingredients by combining an API having poor aqueous solubility with a solution or suspension of one or more pharmaceutically acceptable hydrophilic polymers; (b) forming a wet granulate by combining the granulation ingredients with a mixture of drug dispersion ingredients; and (c) drying the wet granulate to form a dry granulate. In one embodiment, the step of (a) forming a slurry of drug dispersion ingredients by combining an API having poor aqueous solubility with a solution or suspension of one or more pharmaceutically acceptable hydrophilic polymers comprises: (1) dissolving or suspending at least one hydrophilic polymer in a solvent to form a solution, and (2) combining the solution or suspension formed in step (a) with at least one active pharmaceutical ingredient having a solubility in water of less than about 1 mg/ml to form a mixture and blending the mixture to form a slurry. In another embodiment, the step of (b) forming a wet granulate by combining the granulation ingredients with the mixture of drug dispersion ingredients comprises: (3) forming a mixture of at least one diluent and at least one disintegrant; (4) combining the slurry of the at least one active ingredient and the at least one hydrophilic polymer with the mixture formed in step (3), and (5) mixing the mixture of step (4) to form a wet granulate. The processes of combining and/or mixing can be by any mixing or dispersing means as is known in the art. For example, the ingredients can be combined using a twin-shell mixer of the Patterson-Kelly type, a planetary mixer of the Glen type, or a high shear/high intensity or high speed mixer of the Henschel, Lodige/Littleford, or Baker-Perkins types. Use of a low shear mixer is the preferred means of combining ingredients, especially when forming the slurry of the at least one API and the at least one hydrophilic polymer. The wet granulate can be dried, using methods that are well known to those in the art such as, for example, in a tray drier or fluidized bed drier.

The dry granulate may optionally be further processed to alter the particle size distribution of the granulate and to add additional ingredients, such as at least one lubricant, to the granulate. Processes for altering the distribution of particle sizes are well known in the art and include milling, screening and combinations thereof. For example, a Fitzpatrick mill with an appropriate size screen, such as, for example, a 0.5 mm screen can be suitable for use in this step.

A pharmaceutical composition in an oral dosage form, such as tablets or capsules, may be prepared using granulates described above. The dry granulate obtained by the methods described above can be used directly, or can be blended with one or more additional pharmaceutically acceptable excipients prior to use. In one embodiment, the granulate is blended with at least one lubricant prior to use, for example, prior to being compressed into tablets. The dry granulates can be further processed to change the particle size distribution to a desired distribution. The particle size distribution of the granules may be adjusted to affect the dissolution profile or the release rate profile of the active ingredient from the formula. The dry granules may also be blended or combined with one or more additional pharmaceutically acceptable excipients prior to use in the pharmaceutical formulation. These excipients can include excipients described above. In an embodiment, the dry granules are combined with one or more lubricants. The pharmaceutical formulation can further comprise at least one coating. One of ordinary skill in the art would recognize that coatings can be used for a variety of purposes, including providing stability to the dosage form, adjusting the release rate of the API from the dosage form, adjusting the disintegration rate of the dosage form, and providing identification information regarding the dosage form. Such a person would also recognize how to select and use such coatings to achieve the desired effects.

The present invention also relates to a granulate prepared by any of the processes described above.

In order to further illustrate the present invention and the advantages thereof, the following specific examples are given, it being understood that same are intended only as illustrative and in no way limitative. In said examples to follow, all parts and percentages are given by weight, unless otherwise indicated.

Examples 1-3 are representative examples of tablet formulations comprising bicalutamide as the API in granules. The core of the granules comprises bicalutamide as the active ingredient and povidone as the hydrophilic polymer, where the core was formed by a slurry of bicalutamide and an aqueous solution of povidone. Differing amounts of povidone are present in the core. The granules comprises the core in contact with the various excipients indicated in the table as granulation ingredients, where the diluent is lactose monohydrate and the disintegrant is sodium starch glycolate. The amounts of lactose monohydrate and sodium starch glycolate vary between the formulations. In Example 3, povidone is also present as an excipient with lactose monohydrate and sodium starch glycolate in contact with the core. The tablets further comprise a mixture of lubricants comprising colloidal silicon dioxide and a mixture of magnesium stearate/sodium lauryl sulfate which were added to the granulate.

TABLE 1
Examples of Bicalutamide Tablet Formulations:

| | % w/w | | |
|---|---|---|---|
| Ingredients | Example 1 | Example 2 | Example 3 |
| Drug Dispersion Ingredients: | | | |
| Povidone (PLASDONE ® K29/32 or | 5.0 | 10.0 | 3.63 |
| KOLLIDON ® 30) | | | |
| Purified Water, USP¹ | (17.3) | (17.3) | (10.9) |
| Bicalutamide | 41.67 | 41.67 | 41.67 |
| Granulation Ingredients: | | | |
| Lactose Monohydrate | 42.58 | 37.58 | 41.58 |
| Sodium Starch Glycolate | 9.0 | 9.0 | 3.0 |
| Povidone (PLASDONE ® K29/32 or | 0.0 | 0.0 | 8.37 |
| KOLLIDON ® 30) | | | |
| Lubricant Ingredients: | | | |
| Magnesium Stearate/Sodium Lauryl | 1.25 | 1.25 | 1.25 |
| Sulfate (94/6) (STEAR-O- | | | |
| WET ® M) | | | |
| Colloidal Silicon Dioxide | 0.5 | 0.5 | 0.5 |
| Total | 100 | 100 | 100 |

¹Removed during processing and not part of the final tablet weight

Each of these example tablets were prepared using granulates made by the reverse wet granulation process of the present invention. The granulates along with the added lubricants were compressed to form tablets.

### Example 4

Tablets of bicalutamide were prepared by the process described above using the ingredients listed in Table 2, below, in the amounts shown.

TABLE 2
Composition of Bicalutamide Tablets:

| Ingredient Description | mg |
|---|---|
| Bicalutamide | 50.0 |
| Povidone, K29-32/30 | 12.0 |
| Lactose, monohydrate | 45.1 |
| Sodium Starch Glycolate (Explotab/Glycolys/Primojel) | 10.8 |
| Magnesium Stearate/Sodium Lauryl Sulfate | 1.50 |
| (94:6) (Stear- | |
| Colloidal Silicon Dioxide (Cab-O-Sil, M-5) | 0.60 |
| Total Weight of Tablet Before Coating | 120.0 |
| COATING: White Opadry II (Y-22-7719) | 5.0 |
| Total Weight of Coated Tablet | 125.0 |

### Example 5

The API, bicalutamide, and the hydrophilic polymer, povidone, were combined by dissolving povidone (18.0 g) in purified water (62.4 g) to form a solution and adding the solution, with mixing, to a powder of bicalutamide (150 g) in the bowl of a granulator. The mixture was stirred until a dispersion in the form of a slurry was formed. The granulation ingredients, lactose monohydrate (153.3 g) and sodium starch glycolate (32.4 g), where combined in a separate blender with mixing to form a mixture of the granulation ingredients. The mixture of the granulation ingredients were added to the dispersion of povidone, water and bicalutamide with mixing. The mixing was continued until a wet granulate was obtained. The wet granulate was dried in an oven until the desired moisture level was obtained. The dried granulate was then passed through a Fitzmill. These granules (272.4 g) were blended with wettable blend of magnesium stearate/sodium lauryl sulfate (94/6) (3.47 g) (STEAR-O-WET® M produced by Mallinckrodt) and colloidal silicon dioxide (1.39 g) and the resulting blend was compressed into tablets.

### Examples 6 and 7

A comparison of the dissolution of tablets made from granules formed using the standard wet granulation process (Example 6) was made with the tablets made from granules formed using the reverse wet granulation process (Example 7) generally described above in Example 5, using the composition described in Example 2. The dissolution of the tablets of Examples 6 and 7 were evaluated using the FDA recommended dissolution test using USP apparatus II (paddle) with 1000 ml of a 1% aqueous solution of sodium lauryl sulfate at 37° C. with the paddle apparatus rotating at 50 rpm. The results of the tests are summarized in Table 3, below.

TABLE 3
Comparison of Reverse Wet Granulation Process with
Standard Wet Granulation Process for Bicalutamide Tablets:

| | Standard Granulation | Reverse Granulation |
|---|---|---|
| Granulating Process | (Example 6) | (Example 7) |
| Process Variables | | |
| % Water Added | 15 | 15 |
| Mixing Time (min)¹ | 3 | 3 |
| Dissolution | % Dissolved (n = 3) | |
| 5 min | 30 | 27 |
| 15 min | 73 | 72 |
| 30 min | 93 | 96 |
| 45 min | 97 | 101 |
| 60 min | 99 | 103 |
| Granulation Endpoint | Sharp, narrow range | Wide range |
| Dissolution Observations | Uneven erosion with | Uniform erosion with |
| | large dispersed | small dispersed |
| | particles | particles |

¹The mixing time after addition of all material during granulation.

The results of this test indicate that tablets produced using granules formed by the reverse wet granulation process have a wider granulation endpoint range those produced using granules formed by the standard one-step process. In addition, tablets produced using granules formed by the reverse wet granulation process eroded more uniformly than those produced using granules formed by the standard one-step process.

### Example 8

A bioequivalence study was conducted to compare the bioequivalence of bicalutamide tablets comprising granules formed using the reverse wet granulation method with the bioequivalence of commercially available tablets produced using the conventional granulation process (CASODEX® 50 mg tablets). The test used healthy male human volunteers ate a standard FDA breakfast meal 30 minutes prior to administration of a single oral dose of 50 mg of bicalutamide. Blood samples were collected from the volunteers at various times up to approximately 504 hours after dosing. The concentration of bicalutamide in plasma was determined by HPLC/MS (high performance liquid chromatography with mass spectrometric detection). The results of the determination of the blood concentrations were used to calculate the following pharmacokinetic parameters: the maximum concentration in the blood (CPEAK); the time at which the maximum concentration was observed (TPEAK); the elimination rate constant (KEL); the area under the plasma concentration-time curve (AUCL); the area under the plasma concentration-time curve from zero to infinity (AUCI); and the elimination half-life (HALF).

| | A = | B = | LSMEANS | 90% |
|---|---|---|---|---|
| | Reverse Wet | Conventional | Ratio | Confidence |
| Parameter | Granulation | Process | (A:B) | Interval |
| AUCL | 174188.2 | 155593.1 | 1.13 | 104.7%-121.5% |
| (ng · hr/ | (23.8%) | (23.9%) | | |
| mL) | | | | |
| AUCI | 190825.3 | 170507.3 | 1.13 | 103.3%-123.9% |
| (ng · hr/ | (30.3%) | (32.9%) | | |
| mL) | | | | |
| CPEAK | 1110.3 | 1041.0 | 1.06 | 101.1%-111.9% |
| (ng/mL) | (16.5%) | (13.7%) | | |
| KEL | 0.0057 | 0.0062 | — | — |
| (hr⁻¹) | (29.1%) | (28.9%) | | |
| HALF (hr) | 131.3 | 123.1 | — | — |
| | (24.15%). | (35.6%) | | |
| TPEAK | 9.00 | 6.36 | — | — |
| (hr) | (93.5%) | (68.2%) | | |

Values are arithmetic means

These results indicate that tablets comprising granules produced by the reverse wet granulation process are bioequivalent to those produced by the conventional wet granulation process.

### Example 9

A comparison of pharmacokinetic profiles, fasted and fed, in patients dosed with bicalutamide tablets made by the reverse wet granulation (test) process are given below. The methodology used was as described above in Example 8 except for the fasting versus fed states of the test subjects. In both fasted and fed states, a comparison was also made to the profile of commercially available tablets produced using the conventional (reference) granulation process (CASODEX® 50 mg tablets).

Fasting

| | Test | Reference | | |
|---|---|---|---|---|
| Parameter | (n = 50) | (n = 49) | Ratio* | 90% C.I.** |
| AUC0-t (ng × hr/ | 145863.5 | 146808.9 | 0.99 | 92.8%-106.5% |
| mL) | | | | |
| AUC∞ (ng × hr/ | 156603.6 | 156403.3 | 1.00 | 92.4%-108.5 |
| mL) | | | | |
| Cₘₐₓ (ng/mL) | 799.3 | 844.3 | 0.95 | 89.8%-99.9% |

Fed

| | Test | Reference | | |
|---|---|---|---|---|
| Parameter | (n = 48) | (n = 45) | Ratio* | 90% C.I.** |
| AUC0-t (ng × hr/ | 170233.2 | 151635.6 | 1.13 | 104.7%-121.5% |
| mL) | | | | |
| AUC∞ (ng × hr/ | 184321.2 | 163689.2 | 1.13 | 103.3%-123.9% |
| mL) | | | | |
| Cₘₐₓ (ng/mL) | 1096.2 | 1032.0 | 1.06 | 101.1%-111.9% |

Values are Least Squares Geometric Means
*Ratio (A/B) = e^{[LSMEAN of LNA - LSMEAN of LNB]}
**Used Natural Log Transformed Parameter

These results indicate that tablets comprising granules produced by the reverse wet granulation process are bioequivalent to those produced by the conventional wet granulation process when used to treat either patients having fasted before treatment or patients having eaten before treatment.

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A granule for pharmaceutical composition, comprising a core which comprises at least one active pharmaceutical ingredient intimately associated with at least one hydrophilic polymer, wherein the active pharmaceutical ingredient has a solubility in water of less than about 1 mg/ml.

2. The granule of claim 1, further comprising at least one excipient selected from the group consisting of diluents, disintegrants, binders, wetting agents, lubricants, glidants, coloring agents and flavoring agents.

3. The granule of claim 2, wherein the at least one excipient comprises at least one diluent and at least one disintegrant.

4. The granule of claim 1, where the at least one active pharmaceutical ingredient having a solubility in water of less than about 1 mg/ml is selected from the group consisting of anastrozole, aripiprazole, atorvastatin, bicalutamide, candesartan, celecoxib, dutasteride, ezetimibe, fenofibrate, glyburide, meloxicam, oxcarbazepine, raloxifene, rifaximine, rofecoxib, simvastatin, and valdecoxib.

5. The granule of claim 1, where the at least one hydrophilic polymer is selected from the group consisting of polyvinyl pyrrolidone, hydroxypropyl methylcellulose, hydroxypropyl cellulose, polyethylene glycol, hydroxyethyl cellulose, polyethylene oxide, carbomer, polyvinyl alcohol, and/or mixtures thereof.

6. The granule of claim 3, where the at least one diluent is selected from the group consisting of lactose monohydrate, microcrystalline cellulose, calcium phosphate dibasic, sucrose, mannitol, starch, pregelatinized starch, lactose, sorbitol, glucose, fructose, galactose, maltose, isomaltose, aluminum oxide, bentonite, powdered cellulose, kaolin, magnesium carbonate, saponite, and mixtures thereof.

7. The granule of claim 3, where the at least one disintegrant is selected from the group consisting of crospovidone, croscarmellose sodium, sodium starch glycolate, and mixtures thereof.

8. The granule of claim 1, where the granule further comprises at least one lubricant selected from the group consisting of magnesium stearate, sodium lauryl sulfate, colloidal silicon dioxide, calcium stearate, magnesium lauryl sulfate, potassium benzoate, sodium benzoate, talc, zinc stearate, sodium stearyl fumarate and mixtures thereof.

9. The granule of claim 3, further comprising a lubricant.

10. The granule of claim 9, where the at least one diluent is lactose monohydrate, the disintegrant is sodium starch glycolate, and the lubricant is magnesium stearate/sodium lauryl sulfate and colloidal silicon dioxide.

11. A pharmaceutical dosage form, comprising granules of claim 1.

12. A pharmaceutical tablet comprising granules wherein individual granules have a core of bicalutamide intimately associated with povidone, the individual granules further comprising a diluent, a disintegrant and a lubricant.

13. A process for making a granulate, comprising:
(a) forming a slurry of drug dispersion ingredients by combining an active pharmaceutical ingredient having poor water solubility with a solution or suspension of one or more pharmaceutically acceptable hydrophilic polymers;
(b) forming a wet granulate by combining granulation ingredients with a slurry of drug dispersion ingredients; and
(c) drying the wet granulate to form a dry granulate.

14. A process for making a granulate, comprising:
(a) dissolving or suspending at least one hydrophilic polymer in a solvent to form a solution or suspension, respectively,
(b) combining the solution or suspension formed in step (a) with an active pharmaceutical ingredient having a solubility in water of less than about 1 mg/ml to form a mixture and blending the mixture to form a slurry,
(c) forming a mixture of at least one diluent and at least one disintegrant,
(d) combining the mixture formed in step (b) with the mixture formed in step (c),
(e) mixing the mixture of step (d) to form a wet granulate, and
(f) drying the wet granulate to obtain a dry granulate.

15. The process of claim 13, further comprising the step of blending at least one lubricant and/or other excipient with the dry granulate.

16. The process of claim 13, further comprising the step of processing the dry granulate to modified the particle size distribution of the dry granulate.

17. A granulate formed by the process of claim 13.

18. A granulate comprising granules of claim 1.

19. A process for making a pharmaceutical tablet, the process comprising compressing the granules of claim 1 into tablets.

20. A process of making a pharmaceutical capsule, comprising filling a capsule shell with granules of claim 1 to obtain the capsule, said process optional comprising the step of including one or more additional pharmaceutically acceptable excipients.
